(19) United States
(12) Patent Application Publication
(10) Pub. No.: US 2026/0054131 A1
(43) Pub. Date: Feb. 26, 2026

(54) EXERCISE REGIMEN EVALUATION USING CHEMICAL SENSORS

(71) Applicant: Cardiac Pacemakers, Inc., St Paul, MN (US)

(72) Inventors: Pramodsingh Hirasingh Thakur, Woodbury, MN (US); Michael J. Kane, St Paul, MN (US); Ramesh Wariar, Blaine, MN (US); Yingbo Li, Woodbury, MN (US)

(21) Appl. No.: 19/303,581

(22) Filed: Aug. 19, 2025

Related U.S. Application Data
(60) Provisional application No. 63/685,250, filed on Aug. 20, 2024.

(51) Int. Cl. A63B 24/00; A61B 5/0205; A61B 5/024; A61B 5/11; A61B 5/145; A61B 5/1473

(52) U.S. Cl. CPC A63B 24/0075; A61B 5/0205; A61B 5/14514; A61B 5/14539; A61B 5/1473; A61B 5/02438; A61B 5/1118; A61B 5/14546; A61B 2562/0219; A63B 2024/0068

(57) ABSTRACT

Systems, devices, and methods involve approaches for comparing a first set of pH levels measured using a chemical sensor during a first exercise regimen with a second set of pH levels measured using the chemical sensor during a second exercise regimen, determining that the second exercise regimen results in a lower pH deviation than the first exercise regimen, and recommending that the second exercise regimen replace the first exercise regimen.

### CROSS REFERENCE TO RELATED APPLICATION

This application claims priority to Provisional Application No. 63/685,250, filed Aug. 20, 2024, which is herein incorporated by reference in its entirety.

### TECHNICAL FIELD

Instances of the present disclosure relate to using analyte sensing technology for evaluating exercise regimens.

### BACKGROUND

Exercise can improve the health of patients experiencing heart failure.

### SUMMARY

In Example 1, a method includes comparing a first set of pH levels measured using a chemical sensor during a first exercise regimen with a second set of pH levels measured using the chemical sensor during a second exercise regimen, determining that the second exercise regimen results in a lower pH deviation than the first exercise regimen, and recommending that the second exercise regimen replace the first exercise regimen.

In Example 2, the method of Example 1, wherein the chemical sensor is a wearable device that includes needles sized for access to interstitial fluid and a chemical indicator positioned within the needles.

In Example 3, the method of Example 1, wherein the chemical sensor is part of an implantable medical device and comprises a chemical indicator.

In Example 4, the method of Examples 2 or 3, wherein the chemical indicator changes optical properties in response to different pH levels.

In Example 5, the method of any of Examples 2-4, further including: measuring the first set of pH levels and the second set of pH levels based on the changes in the optical properties.

In Example 6, the method of any of Examples 1-5, further including: receiving activity data measured using an acceleration sensor.

In Example 7, the method of Example 6, further including: aligning in time pH data measured by the chemical sensor with the activity data measured using the acceleration sensor.

In Example 8, the method of Example 7, further including: determining a pH-to-activity slope.

In Example 9, the method of Examples 7 or 8, further including: recommending a time period for starting the second exercise regimen based, at least in part, on the pH data and the activity data.

In Example 10, the method of any of Examples 1-9, further including: aligning in time pH data measured using the chemical sensor with heart rate data, determining that the heart rate data and the pH data indicate that a lactate threshold is reached due to musculoskeletal composition, and recommending another exercise regimen that increases muscle mass.

In Example 11, the method of any of Examples 1-9, further including: aligning in time pH data measured using the chemical sensor with heart rate data, determining that the heart rate data and the pH data indicate that exercise intolerance is due to a cardiovascular issue, and recommending another exercise regimen that improves cardiovascular response to exercise.

In Example 12, the method of any of Examples 1-11, further including: comparing a third set of pH levels measured using the chemical sensor during a third exercise regimen with the second set of pH levels, determining that the second exercise regimen results in a lower pH deviation than the third regimen, and recommending that the third exercise regimen not replace the second exercise regimen.

In Example 13, a computer program product comprising instructions to cause one or more processors to carry out the steps of the method of Examples 1-12.

In Example 14, the computer-readable medium having stored thereon the computer program product of Example 13.

In Example 15, the mobile device comprising the computer-readable medium of Example 14.

In Example 16, a system includes a mobile computing device including a processor, memory, and a user interface. The mobile computing device is programmed to: compare a first set of pH levels measured using a chemical sensor during a first exercise regimen with a second set of pH levels measured using the chemical sensor during a second exercise regimen, determine that the second exercise regimen results in a lower pH deviation than the first exercise regimen, and recommend, via the user interface, the second exercise regimen.

In Example 17, the system of Example 16, further including: the chemical sensor, wherein the chemical sensor is a wearable device with needles sized for access to interstitial fluid and a chemical indicator positioned within the needles, wherein the chemical indicator changes optical properties in response to different pH levels.

In Example 18, the system of Example 17, wherein the mobile computing device includes an image sensor.

In Example 19, the system of Example 18, wherein the mobile computing device is programmed to: determine the first pH levels and the second pH levels based, at least in part, on an optical property of the chemical indicator.

In Example 20, the system of Example 16, wherein the mobile computing device includes an image sensor and is programmed to: determine the first pH levels and the second pH levels based, at least in part, on an optical property of a chemical indicator in a digital image.

In Example 21, the system of Example 16, further including: the chemical sensor, wherein the chemical sensor is part of an implantable medical device and comprises a chemical indicator, wherein the chemical indicator changes optical properties in response to different pH levels.

In Example 22, the system of Example 16, wherein the mobile computing device is programmed to: receive activity data measured using an acceleration sensor.

In Example 23, the system of Example 22, further including: the acceleration sensor coupled to a patient.

In Example 24, the system of Example 22, wherein the mobile computing device is programmed to: align in time pH data measured by the chemical sensor with the activity data.

In Example 25, the system of Example 22, wherein the mobile computing device is programmed to: determine a pH-to-activity slope.

In Example 26, the system of Example 22, wherein the mobile computing device is programmed to: recommend a time period for starting the second exercise regimen based, at least in part, on the pH data and the activity data.

In Example 27, the system of Example 16, wherein the mobile computing device is programmed to: align in time pH data measured using the chemical sensor with heart rate data, determine that the heart rate data and the pH data indicate that a lactate threshold is reached due to musculoskeletal composition, and recommend, via the user interface, another exercise regimen that increases muscle mass.

In Example 28, the system of Example 27, further including: a heart rate sensor coupled to a patient and configured to generate the heart rate data.

In Example 29, the system of Example 16, wherein the mobile computing device is programmed to: align in time pH data measured using the chemical sensor with heart rate data, determine that the heart rate data and the pH data indicate that exercise intolerance is due to a cardiovascular issue, and recommend, via the user interface, another exercise regimen that improves cardiovascular response to exercise.

In Example 30, the system of Example 16, wherein the mobile computing device is programmed to: compare a third set of pH levels measured using the chemical sensor during a third exercise regimen with the second set of pH levels, determine that the second exercise regimen results in a lower pH deviation than the third regimen, and recommend, via the user interface, that the third exercise regimen not replace the second exercise regimen.

In Example 31, a method includes: comparing a first set of pH levels measured using a chemical sensor during a first exercise regimen with a second set of pH levels measured using the chemical sensor during a second exercise regimen, determining that the second exercise regimen results in a lower pH deviation than the first exercise regimen, and recommending that the second exercise regimen replace the first exercise regimen.

In Example 32, the method of Example 31, further including: comparing a third set of pH levels measured using the chemical sensor during a third exercise regimen with the second set of pH levels, determining that the second exercise regimen results in a lower pH deviation than the third regimen, and recommending that the third exercise regimen not replace the second exercise regimen.

In Example 33, the method of Example 31, further including: aligning in time pH data measured using the chemical sensor with heart rate data, determining that the heart rate data and the pH data indicate that a lactate threshold is reached due to musculoskeletal composition, and recommending another exercise regimen that increases muscle mass.

In Example 34, the method of Example 31, further including: aligning in time pH data measured using the chemical sensor with heart rate data, determining that the heart rate data and the pH data indicate that exercise intolerance is due to a cardiovascular issue, and recommending another exercise regimen that improves cardiovascular response to exercise.

In Example 35, the method of Example 31, further including: receiving or generating activity data measured using an acceleration sensor and recommending a time period for starting the second exercise regimen based, at least in part, on the pH data and the activity data.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of a chemical sensing system, in accordance with certain instances of the present disclosure.

FIG. 2 shows a block diagram of a method for use with one or more components of a chemical sensing system, in accordance with certain instances of the present disclosure.

FIG. 3 shows a schematic illustration of a computing device, in accordance with certain instances of the present disclosure.

FIGS. 4 and 5 show block diagrams of methods for use with one or more components of a chemical sensing system, in accordance with certain instances of the present disclosure.

FIGS. 6-8 show different views of various portions of a wearable chemical sensing device, in accordance with certain instances of the present disclosure.

FIGS. 9 and 10 show different views of various portions of an implantable medical device, in accordance with certain instances of the present disclosure.

FIG. 11 shows a block diagram with additional details of the computing device of FIG. 3, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

A person experiencing heart failure conditions may have a low tolerance for exercise. During exercise, a person's blood lactate level increases and may reach the person's lactate threshold, which will cause the person to be unable to continue exercising. As such, a low lactate threshold results in a low tolerance for exercise.

However, exercise can ultimately improve the person's condition and therefore quality of life and lifespan. Depending on the individual, a person may be able to perform certain exercises for a longer period of time and/or at a higher intensity before reaching their lactate threshold. Over time, this can increase their lactate threshold, which allows the person to exercise for an even longer period of time and/or at a higher intensity—which can improve the person's condition.

Certain instances of the present disclosure are accordingly directed to approaches (e.g., systems, methods, devices) that use analyte sensing technology (and, in certain instances, multiple sensing technologies) for evaluating and recommending exercise regimens. In some instances, the analyte is lactate. In other instances, the analyte is a person's pH level—which decreases as lactate levels increase.

### Analyte Sensing System

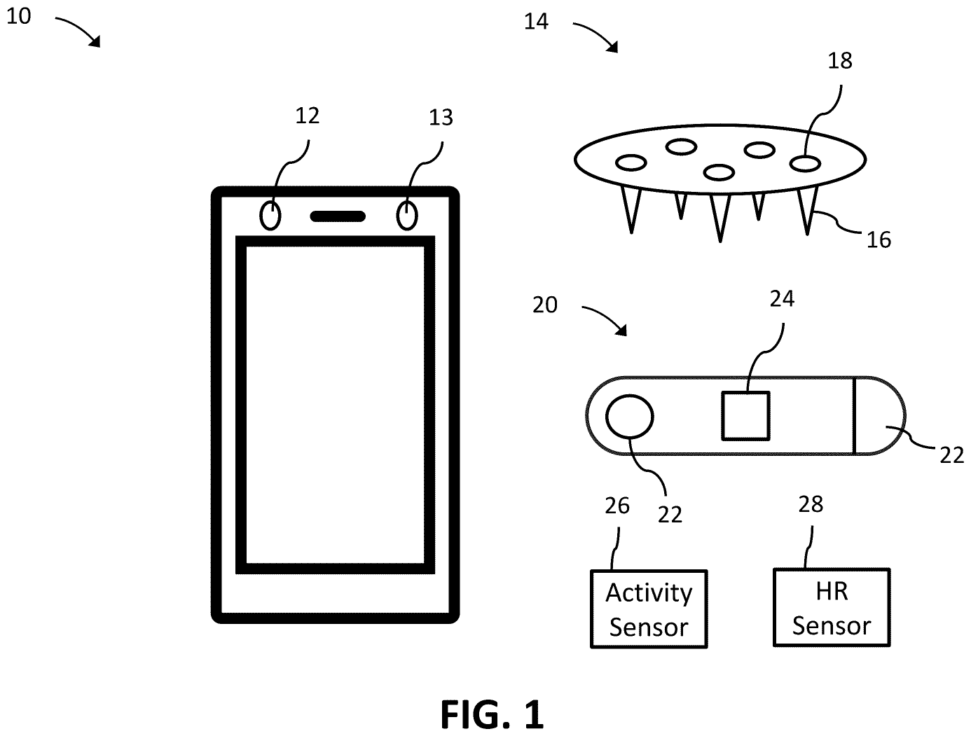

FIG. 1 shows a chemical sensing system 10 (hereinafter “the system 10” for brevity) with schematic representations of two sets of components that can be used to measure pH levels and/or analyte levels (e.g., concentrations). Typically, measuring a patient's analyte levels requires drawing multiple blood samples from a patient at a clinic and processing the blood samples at a laboratory. The components of the chemical sensing system 10 can be used to measure pH levels and analyte concentrations using a wearable device or an implantable medical device. Although the chemical sensors shown in FIG. 1 and described below are optical-based chemical sensors, other types of chemical sensor approaches can be used (e.g., electrical-based chemical sensors such as those that use electrodes and measure potential differences, and the like).

For the wearable approach, the system 10 can include a device (e.g., a mobile computing device described further herein) with an image sensor 12 and a chemical sensing device 14. The image sensor 12 (e.g., a charge coupled device, a complementary metal oxide semiconductor, or other devices that can capture an image) can be part of a camera, smart phone, or other device able to capture an image (e.g., a digital image). In certain instances, the image sensor 12 and the chemical sensing device 14 are integrated into a single device, and in other instances the image sensor 12 and the chemical sensing device 14 are separate devices. In instances where the image sensor 12 is part of a mobile computing device such as a smart phone, the smart phone can store, operate, or otherwise access a program (e.g., a phone application) that processes an image (of the chemical sensing device 14) taken by the image sensor 12 and determines estimates of pH and/or one or more analyte concentrations of the patient. In other instances, the image sensor 12 is part of a dedicated readout device or part of a camera. The system 10 can include one or more light sources 13, which can be part of the same device as the image sensor 12 or which can be part of a separate component. The one or more light sources 13 can generate light (e.g., emit visible light, ultraviolet light, monochromatic light (red, green, blue)).

The chemical sensing device 14 can be a wearable device (e.g., an exterior device and not an implantable device) such as a device that includes (or is part of) a strap (e.g., an armband strap), a patch (e.g., a torso patch), or another type of device that can be coupled to a patient's skin. For simplicity, the chemical sensing device 14 is hereinafter referred to as the “patch 14” although other types of wearable devices can use the chemical sensing technology described herein.

In certain instances, the patch 14 is a transdermal patch that includes a mechanism (e.g., needles 16) to access a patient's interstitial fluid. For example, multiple needles 16 (e.g., microneedles) can be sized to access a patient's interstitial fluid. The patch 14 can also include multiple chemical indicators 18, each of which changes optical properties (e.g., fluorimetric properties, colorimetric properties) with changes in pH or concentration of a certain analyte in the interstitial fluid. As described in more detail herein, the image sensor 12 can be used to capture an image (e.g., a digital image) of the chemical indicators 18, and the image can be processed and analyzed to determine respective pH and concentrations of targeted analytes. In certain instances, the patch 14 includes one type of chemical indicator 18 (e.g., to help determine pH levels or concentration of one type of analyte), but in other instances the patch 14 includes multiple types of chemical indicators.

For the implantable approach, the system 10 can include an implantable medical device 20, which includes one or more electrodes 22 and a chemical sensor assembly 24. The electrodes 22 can comprise a conductive material and be configured to sense cardiac activation signals. The chemical sensor assembly 24 can include a sensing element with a polymeric matrix permeable to analytes. The sensing element can include an interior volume with various chemical indicators (e.g., beads for detecting an ion concentration of a bodily fluid when implanted in the body disposed within an interior volume). Analytes can diffuse through an outer barrier layer and onto and/or into the chemical indicators where the analytes can bind with ion selective sensors to produce an optical response (e.g., a change in optical properties such as a change in concentration, a fluorimetric response, a colorimetric response). The optical response can be monitored and used to estimate analyte levels. The estimated analyte levels can be used by a computing device to monitor and evaluate a person's kidney and/or cardiac performance.

In certain instances, the system includes multiple chemical sensor assemblies. Using a multi-sensor approach, measurements can be compared to or used in conjunction with each other. For example, a differential pH level could be calculated based on pH levels measured by a chemical sensor assembly coupled to a major muscle group and pH levels measured by chemical sensor assembly positioned in subcutaneous tissue. Using this approach, local pH at the muscle can be compared to systemic or circulating pH in the bloodstream.

The system 10 can also include additional sensors such as an activity sensor 26 (e.g., an acceleration sensor such as a single-axis or multi-axis accelerometer) and a heart rate sensor 28 (e.g., pulse sensor, ECG sensor, PPG sensor).

### Methods

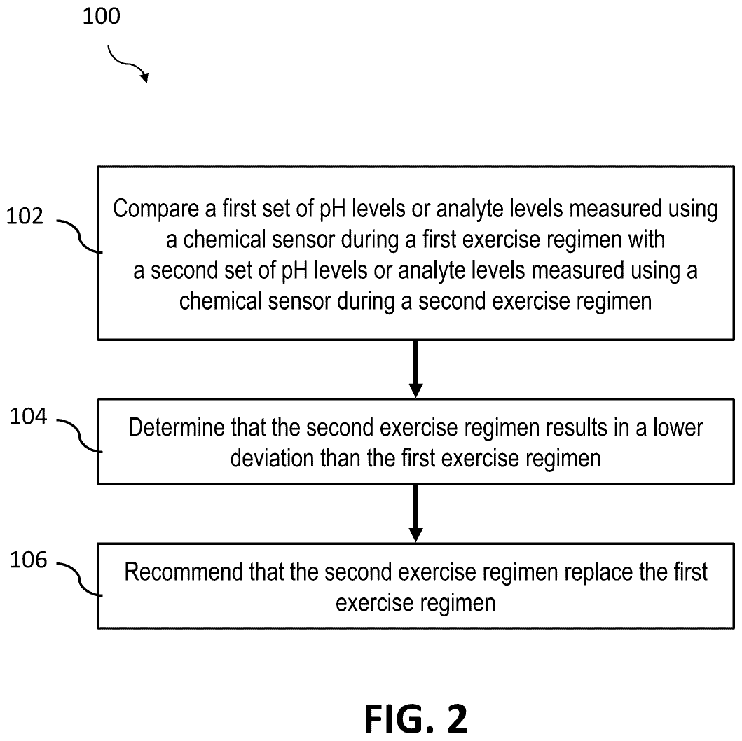

FIG. 2 outlines a method that can be used in connection with one or more components of the system 10 of FIG. 1 or other components described herein.

FIG. 2 shows a block diagram of a method 100 for identifying an exercise regimen that can improve a person's exercise tolerance over time. In certain instances, the method 100 is carried out by one or more computing devices such as a mobile phone, a tablet, a laptop computer, a desktop computer, a server. For example, the method 100 could carried out by an application (e.g., a phone app) that is downloadable to the device, operated on the device, and accessible by a patient, their physician, their clinic, and the like.

The method 100 includes comparing a first set of pH levels or analyte levels measured using a chemical sensor during a first exercise regimen with a second set of pH levels or analyte levels measured using the chemical sensor during a second exercise regimen (block 102 in FIG. 2). The chemical sensor can be the types of chemical sensors described in connection with FIG. 1 and herein. As such, a chemical indicator that is part of the chemical sensor can be in communication with a person's blood such that one or more optical properties of the chemical indicator can be monitored to determine a pH level or concentration of a given analyte.

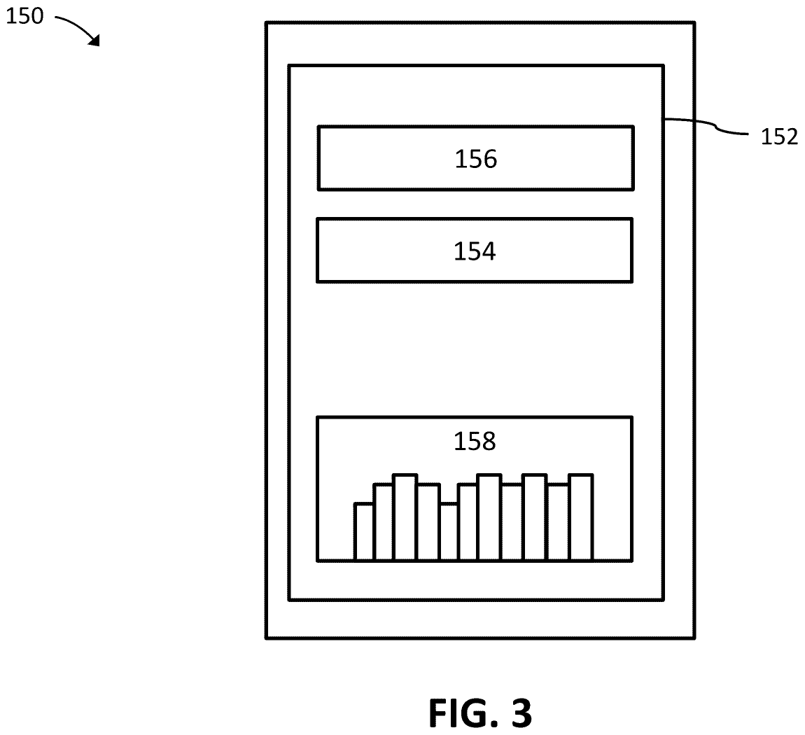

In certain instances, the first exercise regimen is initially selected by the patient or their physician. The first exercise regimen can be considered to be a baseline exercise regimen. In certain embodiments, the instructions for the exercise regimen can be displayed on a user interface of a computing device. FIG. 3 shows an example computing device 150 such as a mobile phone or tablet that includes a user interface 152. The device 150 can operate an application such that the user interface 152 is displayed with various screens, icons, and/or buttons to help the user navigate and use the application. One or more pictures, videos, and/or text passages 154 can be used to display the instructions for the first exercise regimen to the user.

In certain instances, after the user attempts to complete the first regimen, the results of the measurements (e.g., pH measurements) can be sent to the device 150 (or another device in a chemical sensing system).

In certain instances, the second exercise regimen is selected by the patient or their physician. For example, the second exercise regimen can be selected based on the patient's personal preference for completing the exercise (as compared to the baseline exercise regimen). In certain instances, the application operated by the device 150 can include a library or database of selectable exercise regimens, and each selectable exercise regimen can be associated with its set of own pictures, videos, and/or text passages that can be used to display the instructions for the selected exercise regimen. In certain instances, after the user attempts to complete the second regimen, the results of the analyte measurements can be sent to the device 150 (or another device in a chemical sensing system).

In some instances, the analyte being monitored is lactate while in other instances pH levels are monitored. As noted above, a person's pH level decreases as their lactate level increases. As such, pH levels can be used as a proxy for lactate levels. When a person is in a sedentary position, a typical pH level is approximately 7.5. When a person begins to exercise, their pH level will begin drop (e.g., deviate from their normal level). As the person continues to exercise and get closer to their lactate threshold (e.g., exercise intolerance level), the pH levels continue to decrease. In certain instances, once a person's pH level lowers a certain amount (e.g., by 5% or more, 7.5% or more, 10% or more) or reaches a specific level (e.g., 7.1 or less), it is likely that the person's lactate level has increased, and they have reached their lactate threshold. In certain instances, the thresholds can be based on a person's individual feedback. For example, a person could provide feedback (e.g., via a mobile phone application) about how that person feels at one or more points during an exercise regimen and therefore at one or more different pH levels. The feedback could be used to set a person's individual threshold because each person may tolerate pH levels differently.

To compare the effectiveness of the baseline exercise regimen with the second exercise regimen, the pH levels and/or analyte levels can be monitored and the measurements compared. As one example, the method 100 can include determining that the second exercise regimen results in a lower pH deviation than the first exercise regimen (block 104 in FIG. 2). Put another way, it can be determined whether the second exercise regimen results in a less drastic change (e.g., in absolute terms or over time) from the patient's prior pH level or from a predetermined pH level (e.g., 7.5). If it takes the person longer to reach a lower analyze threshold (whether a percentage decrease or specific level) during the second exercise regimen, the second exercise regimen can be considered to be more effective. Another way to determine pH deviation includes comparing how well a patient recovers from a given exercise regime (e.g., how quick does the pH revert back to the patient's resting state pH level).

As a result, the method 100 can further include recommending that the second exercise regimen replace the first exercise regimen (block 106 in FIG. 2) as the preferred exercise regimen. For example, referring back to FIG. 3, an icon 156 in the user interface 152 can be displayed as a higher priority exercise regimen compared to the icon 154 representing the first exercise regimen. As another example, the user interface 152 can display a passage of text, a graphic, a sound, a video, and the like to indicate that the second regimen is recommended over the first exercise regimen.

This process of testing different exercise regimens can be continued and repeated to find more effective exercise regimens for a particular individual. For example, a third set of pH or analyte measurements associated with a third exercise regimen can be received and compared to measurements from the second set of measurements. If the third exercise regimen is less effective than the second exercise regimen, the second exercise regimen can remain as the recommended exercise regimen for that individual. This process allows for an exercise regimen (or set of regimens) to be customized for individual patients, who can focus on testing regimens that they prefer to perform or can better tolerate—which can increase the likelihood that the patient will follow the regimens and improve their tolerance for exercise (e.g., their lactate threshold).

Referring back to FIG. 1, the system 10 includes additional sensors besides chemical sensors such as one or more activity sensors 26 and/or one or more heart rate sensors 28.

The activity sensor 26 can measure a person's activity, for example, by measuring acceleration (e.g., in terms of mG). The activity sensor 26 can be used to generate activity data (e.g., acceleration measurements as a function of time) throughout the day. In certain instances, the activity sensor 26 can be part of a device coupled to the patient (e.g., a watch, a mobile phone, a ring) or can be part of exercise equipment (e.g., a bike, a treadmill, a rowing machine).

In certain instances, data generated by the activity sensor 26 can be time-aligned with the analyte data from the chemical sensor assembly 24, and the combined data can be used (e.g., by the computing device 150 of FIG. 3) to make determinations, recommendations, etc.

As one example, based on the activity data and the pH/analyte data, the computing device 150 can recommend a time period at which an exercise regimen should be performed. More specifically, if a person's pH/analyte level is at a given level (or predicted to be at a given level), the computing device 150 can recommend to the patient via the user interface 152 (e.g., via an alert, alarm, notification, graphic, text passage) that an exercise regimen be performed. Similarly, based on the activity data and the pH/analyte data measured during an exercise regimen or just throughout the day (e.g., during activities of daily living), the computing device 150 can generate a pH-to-activity slope or analyte-to-activity slope. And the slope can be used to determine a time period to recommend for performing an exercise regimen. Alternatively, the computing device 150 can be programmed to generate a recommendation at preselected date and time. The slope (or another factor) could be used to determine a patient's response to exercise to determine progression or worsening heart failure conditions.

In certain instances, data generated by the heart rate sensor 28 can be time-aligned with the pH/analyte data from the chemical sensor assembly 24, and the combined data can be used (e.g., by the computing device 150 of FIG. 3) to make determinations, recommendations, etc.

As one example, based on the heart rate data and the pH/analyte data during exercise, the computing device 150 can determine whether the patient is reaching their lactate threshold (e.g., based on pH levels) while their heart rate is low (e.g., by comparing to the patient's resting heart rate or a predetermined heart rate). Having a low heart rate while reaching a lactate threshold can indicate that the patient's exercise intolerance is due to peripheral/musculoskeletal issues (e.g., musculoskeletal composition). In response, the computing device 150 can recommend an exercise regimen targeted towards preserving or increasing muscle mass.

As another example, based on the heart rate data and the pH/analyte data during exercise, the computing device 150 can determine whether the patient is not reaching their lactate threshold (e.g., based on pH levels) despite their heart rate being high (e.g., by comparing to the patient's resting heart rate or a predetermined heart rate). Having a high heart rate while failing to reach a lactate threshold can indicate that the patient's exercise intolerance is due to cardiovascular issues. In response, the computing device 150 can recommend an exercise regimen targeted towards improving the patient's cardiovascular response to exercise.

Any of the data described herein-whether historical or in real-time-can be displayed on the user interface 152 of the computing device 150. For example, the user interface 152 can be arranged to include a window 156 in which one or more sets of data can be displayed (e.g., levels as a function of time).

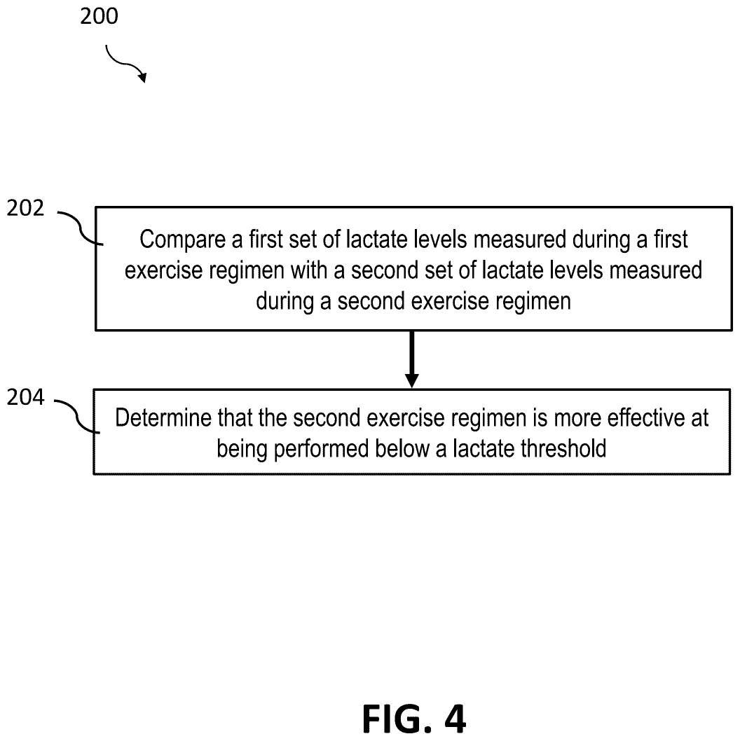

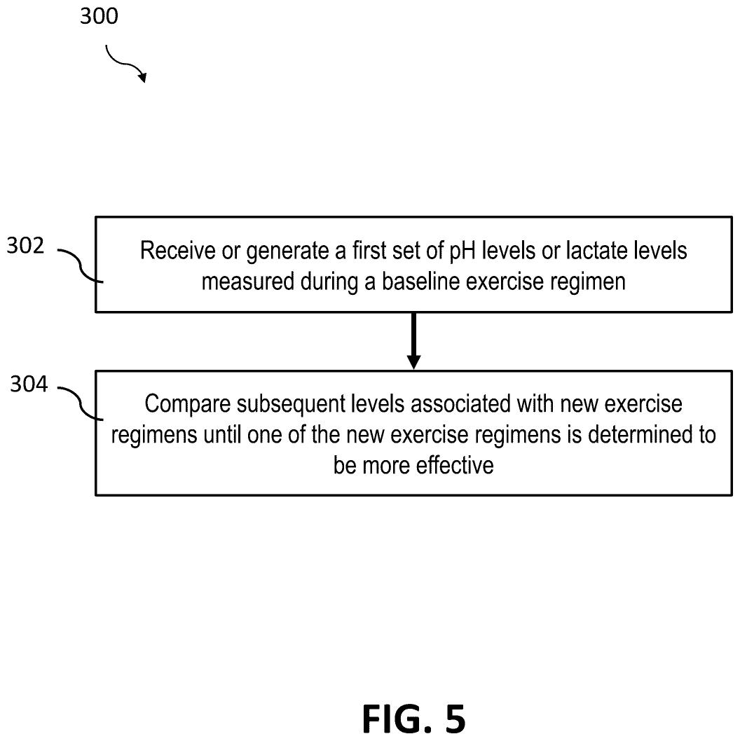

FIGS. 4 and 5 outline method that can be used in connection with one or more components of the system 10 of FIG. 1. In certain instances, the methods are carried out by one or more computing devices such as a mobile phone, a tablet, a laptop computer, a desktop computer, a server. For example, the methods could be carried out by an application (e.g., a phone app) that is downloadable to the device, operated on the device, and accessible by a patient, their physician, their clinic, and the like.

FIG. 4 shows a block diagram of a method 200 for identifying an exercise regimen that can improve a person's exercise tolerance over time. As noted above, in some instances, the analyte being monitored is lactate while in other instances pH is monitored. In the method 200 of FIG. 4, lactate is measured instead of pH.

The method 200 includes comparing a first set of lactate levels measured during a first exercise regimen with a second set of lactate levels measured during a second exercise regimen (block 202 in FIG. 4). The method 200 includes determining that the second exercise regimen is more effective at being performed below a lactate threshold (block 204 in FIG. 4). In response, the second exercise regimen can be recommended to replace the first exercise regimen. In certain instances, the lactate levels are measured using lactate oxidase.

FIG. 5 shows a block diagram of a method 300 for identifying an exercise regimen that can improve a person's exercise tolerance over time. The method 300 includes receiving or generating baseline levels (e.g., pH levels, lactate levels) measured during a baseline exercise regimen (block 302 in FIG. 5). The method 300 further includes comparing subsequent levels associated with new exercise regimens until one of the new exercise regimens is determined to be more effective (e.g., by improving a person's exercise tolerance over time) (block 304 in FIG. 4). Once one of the new exercise regimens is determined to be more effective, that exercise regimen can be identified as a preferred exercise regimen.

Below are additional details of different types of chemical sensors that can be used in connection with identifying an exercise regimen that improves a person's exercise tolerance over time.

### Wearable Chemical Sensor

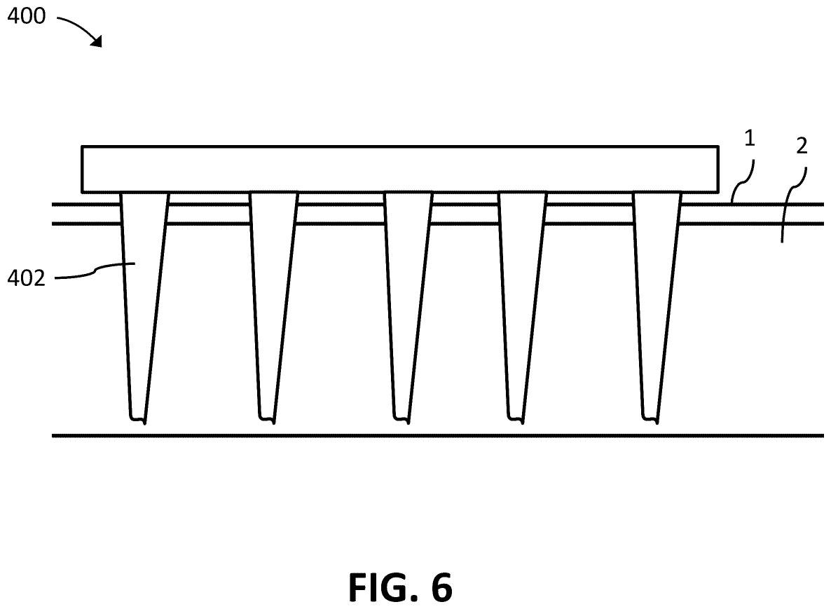

FIG. 6 shows a schematic side view of a wearable chemical sensing device 400. For simplicity, the device 400 is hereinafter referred to as the “patch 400” although other types of wearable devices can use the chemical sensing technology described herein. The patch 400 can be coupled to the patient's skin 1 such that needles 402 pierce through the outer layer of skin 1 and extend into the patient's interstitial fluid space 2. The needles 402 can have openings that are exposed to the patient's interstitial fluid (and therefore analytes within the patient's interstitial fluid).

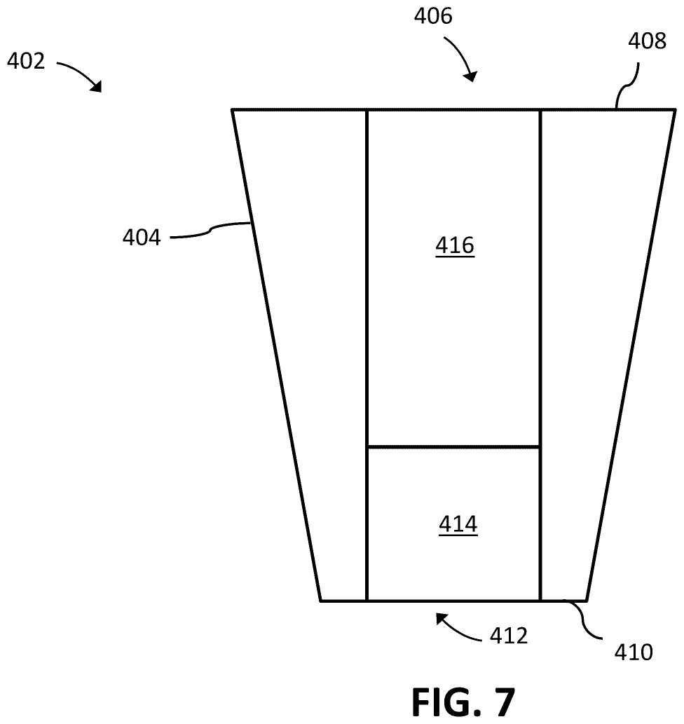

FIG. 7 shows a schematic side view of one of the needles 402 of the patch 400. In certain instances, the needles 402 are hollow needles such that each needle 402 includes an outer needle structure 404 that surrounds an opening 406 (e.g., a central thru-hole within the needle 402). The opening 406 can extend from a proximal end 408 of the needle 402 to a distal end 410 of the needle 402. An aperture 412 is located at or near the distal end 410 of the needle 402 such that the opening 406 is exposed to interstitial fluid.

Also at or near the distal end 410 of the needle 402 is a membrane 414 (e.g., a diffusion membrane) that is positioned within the needle 402. The membrane 414 protects tissue from direct interaction or exposure to a chemical indicator 416 that is also positioned within the needle 402. The membrane 414 can be formed from a permeable material, such as an ion permeable polymeric matrix material. In some instances, the membrane 414 can be permeable to sodium ions, potassium ions, hydronium ions, creatinine, urea, and various additional analytes. As referenced above, the cover membrane of the sensing element can be formed of a permeable material. In some embodiments, the cover membrane can be formed from an ion-permeable polymeric matrix material. Suitable polymers for use as the ion-permeable polymeric matrix material can include, but are not limited to, polymers forming a hydrogel. Hydrogels herein can include homopolymeric hydrogels, copolymeric hydrogels, and multipolymer interpenetrating polymeric hydrogels. Hydrogels herein can specifically include nonionic hydrogels. In certain instances, the membrane 414 includes an active agent disposed therein including, but not limited to anti-inflammatory agents, angiogenic agents, and the like.

The particular type (e.g., type of ion selectivity) and length of membrane can vary by needle 402. For example, one set of needles 402 can include a membrane 414 that is permeable to one type of ions, while another set of needles 402 includes a membrane 414 that is permeable to another type of ions, and so on. In other examples, the membrane 414 is agnostic to a particular type of ion. The membrane 414 is positioned such that analytes must pass through the membrane 414 before reaching the chemical indicator 416. The membrane 414 material used will affect how fast an analyte travels between interstitial fluid and the chemical indicator 416.

The chemical indicator 416 comprises a material that changes properties (e.g., optical properties such as color) with changes in pH levels or concentration of a given analyte. In certain instances, color of the chemical indicator 416 comprises the sum of the absorption, transmission, reflectance, and fluorescence properties of the chemical indicator material. Put another way, the chemical indicator 416 can comprise a material that changes optical properties with changes in concentration of a given analyte—and such optical properties can be measured by analyzing an image of the chemical indicator 416. In certain instances, the chemical indicator 416 has a minimum thickness or height along a longitudinal axis of a needle of 0.15-0.60 mm (e.g., 0.50-0.60 mm). In certain instances, the chemical indicator 416 comprises a slurry or a film.

In certain instances, the chemical indicator 416 is formed of a lipophilic indicator dye (e.g., a lipophilic fluorescent indicator dye or a lipophilic colorimetric indicator dye). Lipophilic indicator dyes can include, but are not limited to, ion selective sensors such as ionophores or fluorophores. In certain instances, ionophores can include sodium-specific ionophores, potassium-specific ionophores, calcium-specific ionophores, magnesium-specific ionophores, and lithium-specific ionophores. In certain instances, fluorophores can include lithium-specific fluorophores, sodium-specific fluorophores, and potassium-specific fluorophores.

Compositions of the chemical indicator 416 can include components (or response elements) that are configured for a colorimetric response, a photoluminescent response, or another optical sensing modality. For example, the chemical indicator 416 can include an element that changes color based on binding with or otherwise complexing with a specific chemical analyte. In some instances, the chemical indicator 416 can include a complexing moiety and a colorimetric moiety. Those moieties can be a part of a single chemical compound (e.g., a non-carrier-based system) or can be separated on two or more different chemical compounds (e.g., a carrier-based system). The colorimetric moiety can exhibit differential light absorbance on binding of the complexing moiety to an analyte.

Some of the chemical indicators 416 may not require a separate compound to both complex an analyte of interest and produce an optical response. By way of example, in some instances, the response element can include a non-carrier optical moiety or material wherein selective complexation with the analyte of interest directly produces either a colorimetric or fluorescent response. As an example, a fluoroionophore can be used and is a compound including both a fluorescent moiety and an ion complexing moiety. As merely one example, (6,7-[2.2.2]-cryptando-3-[2″-(5″-carboethoxy)thiophenyl]coumarin, a potassium ion selective fluoroionophore, can be used (and in some cases covalently attached to polymeric matrix or membrane) to produce a fluorescence-based K+ non-carrier response element. An exemplary class of fluoroionophores are the coumarocryptands. Coumarocryptands can include lithium specific fluoroionophores, sodium specific fluoroionophores, and potassium specific fluoroionophores. For example, lithium specific fluoroionophores can include (6,7-[2.1.1]-cryptando-3-[2″-(5″-carboethoxy)furyl]coumarin. Sodium specific fluoroionophores can include (6,7-[2.2.1]-cryptando-3-[2″-(5″-carboethoxy)furyl]coumarin. Potassium specific fluoroionophores can include (6,7-[2.2.2]-cryptando-3-[2″-(5″-carboethoxy)furyl]coumarin and (6,7-[2.2.2]-cryptando-3-[2″-(5″-carboethoxy)thiophenyl]coumarin.

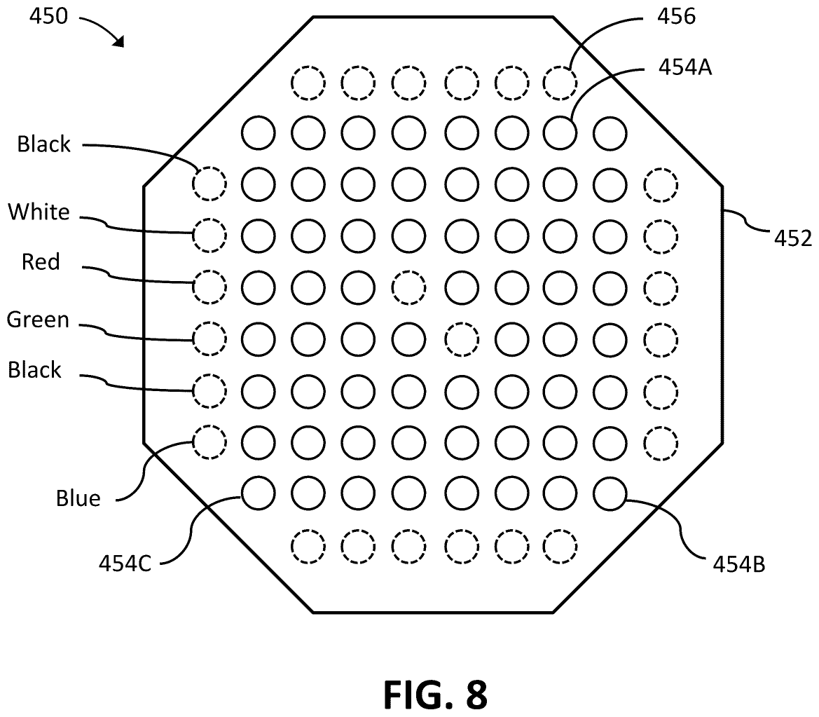

FIG. 8 show a top view of a patch 450. The arrangement shown in FIG. 8 can be used in connections with the needles, membranes, chemical indicators, and layers, etc., described above with respect to the patch 400. The view shown in FIG. 8 is the type of view of a patch that an image sensor would capture in a digital image while the patch is coupled to the patient. The digital image can capture the colors of the chemical indicators (and color references) such that the colors can be analyzed to determine concentrations of one or more analytes of the patient.

FIG. 8 shows a patch 450, which includes a window 452 through which various components of the patch 100 can be viewed. In particular, the window 452 allows chemical indicators 454A-C of the patch 450 to be viewed. The chemical indicators 454A-C can be positioned in needles (e.g., hollow needles) such as the needles described above. As such, each chemical indicator 454A-C can be associated with its own needle.

A first set of needles can include a first type of chemical indicator 454A such as a chemical indicator that changes in color with changes in pH levels. A second set of needles can include a second type of chemical indicator 454B such as a chemical indicator that changes in color with changes in concentration of a second analyte (e.g., sodium). A third set of needles can include a third type of chemical indicator 454C such as a chemical indicator that changes in color with changes in concentration of a third analyte (e.g., potassium). The respective colors of the chemical indicators can be used to estimate the respective pH levels or concentrations of analytes in a patient's interstitial fluid.

In certain instances, each of the first type of chemical indicators 454A are positioned near or next to each other, each of the second type of chemical indicators 454B are positioned near or next to each other, and so on. The overall number of chemical indicators (and therefore the number of needles) and the number of different sets of types of chemical indicators on a given patch can be fewer or greater than that shown in FIG. 8. For example, the patch 450 could include a single type of chemical indicator selected for a single type of analyte. The relative positions of the chemical indicators 454A-C can vary from that shown in FIG. 8, and the specific shape of the chemical indicators 454A-C (as seen from a top view) can vary from the circular shapes shown in FIG. 8.

The patch 450 can also include color references 456. The color references 456 are shown in dotted lines in FIG. 8. The color references 456 can help with calibrating, correcting, and/or processing the digital image of the chemical indicators 454A-C such that an accurate estimate of the color of the chemical indicators 454A-C can be determined. For example, because the color of the color references 456 is known, the color of the chemical indicators 454A-C can be more accurately estimated as the patch 450 is positioned in different lighting (e.g., in direct sunlight, in a shadow, partially shaded, and the like). As such, the color references 456 can act as a color index or reference point for correcting for changes in color caused by ambient light.

In certain instances, some of the color references 456 are black, others white, others red, others green, others blue. Although most of the color references 456 in FIG. 8 are shown around a perimeter of the patch (e.g., with the chemical indicators 454A-C positioned within the perimeter), other positions and arrangements of the color references 456 can be utilized in the patch 450. The overall number and the specific shape of the color references 456 (as seen from a top view) can vary from the circular or dot shape shown in FIG. 8.

Using the patches described herein, analyte concentrations can be estimated. For example, a digital image of a patch attached to a patient can be taken by a camera and an analyte concentration can be estimated based on a color of one or more chemical indicators. In certain instances, estimating the analyte concentrations involves calculating an analyte concentration for multiple chemical indicators and then applying a mathematical operation (e.g., averaging, voting) to determine the respective analyte concentrations. The analyte concentration estimations can be further based on corrections that are determined using color reference sections of the patch. Each set or grouping of chemical indicators from the digital image can be processed and their respective colors compared to a table, library, mapping, index, etc. that associates a given color of chemical indicator to a given concentration level. In certain instances, the process of estimating analyte concentrations is carried out by an application stored on and operated by a smart phone. In other instances, some or all steps can be carried out by a server or other computing system besides a smart phone that can access digital images of a patch and be programmed to determine estimated analyte concentration levels based on colors of chemical indicators shown in the digital image.

U.S. patent application Ser. No. 18/774,681 describes additional details of a wearable chemical sensing system and is herein incorporated by reference in its entirety.

### Implantable Chemical Sensor

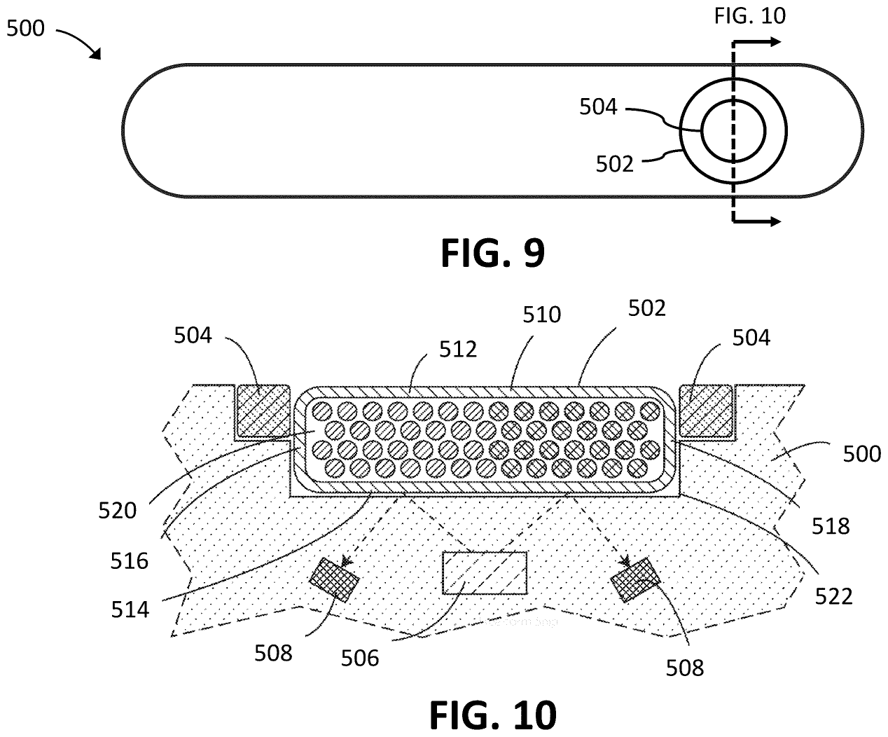

FIGS. 9 and 10 show an implantable medical device (IMD) 500 with a chemical sensor assembly.

FIG. 9 shows a top-down view of the IMD 500 with a sensing element 502 and an active agent eluting material 504 (or active agent eluting matrix) disposed around the outer perimeter of sensing element 502. In some instances, the active agent eluting material 504 forms a ring structure around the outer perimeter of sensing element 502. It will be appreciated that the sensing element 502 and the active agent eluting material 504 can have different geometric shapes and sizes.

FIG. 10 shows a cross-sectional view of a portion of the IMD 500 and its chemical sensor assembly. The IMD 500 includes an optical excitation assembly 506 and optical detection assembly 508. The sensing element 502 can include an outer barrier layer 510 formed, in full or in part, from a permeable material, such as an ion permeable polymeric matrix material. The outer barrier layer 510 can form a top 512, a bottom 514, and opposed sides 516 and 518 to surround an interior volume 520 of the sensing element 502. In certain instances, at least the top 512 of outer barrier layer 510 is permeable to sodium ions, potassium ions, hydronium ions, creatinine, urea, and the like. The outer barrier layer 510 can also include an active agent disposed therein including, but not limited to anti-inflammatory agents, angiogenic agents, and the like. The housing of the IMD 500 can include a recessed pan 522 into which the sensing element 502 fits. In some embodiments, implantable housing can define an aperture occluded by a transparent member. The transparent member can be a glass (including but not limited to borosilicate glasses), a polymer or other transparent material. The aperture can be disposed at the bottom of the recessed pan. The aperture can provide an interface allowing for optical communication between sensing element and the optical excitation and optical detection assemblies. It will be appreciated that outer barrier layer, or portions thereof, can be made from a transparent polymer matrix material to allow for optical communication between the sensing element 502 and optical excitation 506 and optical detection 508 assemblies.

The optical excitation assembly 506 can be designed to illuminate the sensing element 502. The optical excitation assembly 508 can include a light source such as a light emitting diode (LED), vertical-cavity surface-emitting lasers (VCSELs), electroluminescent (EL) devices, and the like. The optical detection assembly 508 can include a component selected from the group consisting of a photodiode, a phototransistor, a charge-coupled device (CCD), a junction field effect transistor (JFET) optical sensor, a complementary metal-oxide semiconductor (CMOS) optical sensor, an integrated photo detector integrated circuit, a light to voltage converter, and the like.

Various indicator beads can be positioned in the interior volume 520. The indicator beads can be used for detecting an ion concentration of a bodily fluid. For example, the indicator beads can include a polymeric support material and one or more ion selective sensing components as described more fully below. Analytes such as potassium ion, sodium ion, hydronium ion, and the like, can diffuse through the top of the outer barrier layer and onto and/or into the indicator beads where they can bind with the ion selective sensors to produce a change in optical properties (e.g., a fluorimetric response, a colorimetric response).

U.S. Patent App. Pub. No. 2018/0344218 describes additional details of an implantable medical device with a chemical sensor assembly and is herein incorporated by reference in its entirety.

### Computing Device

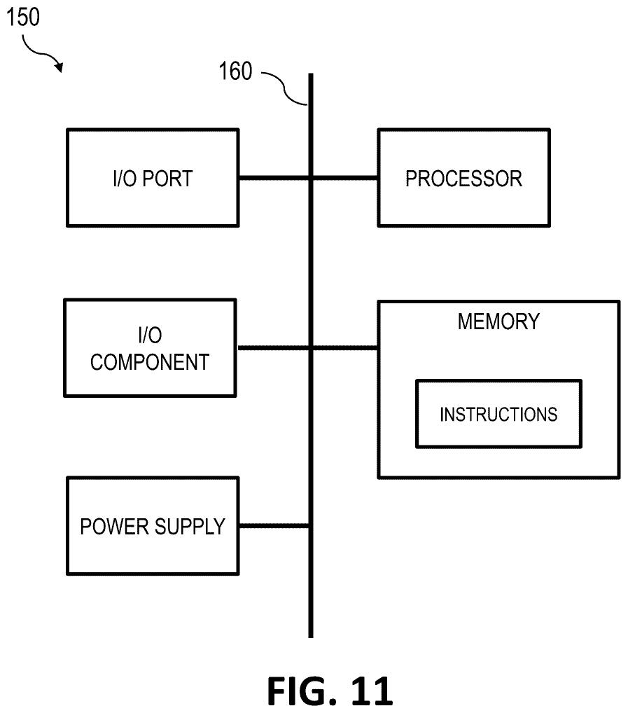

FIG. 11 is a block diagram depicting additional details of the computing device 150 shown in FIG. 3. The computing device 150 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like.

In instances, the computing device 150 includes a bus 160 that, directly and/or indirectly, couples one or more of the following devices: a processor, a memory, an input/output (I/O) port, an I/O component, and a power supply. Any number of additional components, different components, and/or combinations of components may also be included in the computing device 150.

The bus 160 represents what may be one or more busses (such as, for example, an address bus, data bus, or combination thereof). Similarly, in instances, the computing device 150 may include a number of processors, a number of memory components, a number of I/O ports, a number of I/O components, and/or a number of power supplies. Additionally, any number of these components, or combinations thereof, may be distributed and/or duplicated across a number of computing devices.

In instances, the memory includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory stores computer-executable instructions for causing the processor to implement aspects of instances of components discussed herein and/or to perform aspects of instances of methods and procedures discussed herein. The memory can comprise a non-transitory computer readable medium storing the computer-executable instructions.

The computer-executable instructions may include, for example, computer code, machine-useable instructions, and the like such as, for example, program components capable of being executed by one or more processors (e.g., microprocessors) associated with the computing device 150. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions may be configured to be executed by the processor and, upon execution, to cause the processor to perform certain processes. In certain instances, the processor, memory, and instructions are part of a controller such as an application specific integrated circuit (ASIC), field-programmable gate array (FPGA), and/or the like. Such devices can be used to carry out the functions and steps described herein.

The I/O component may include a presentation component configured to present information to a user such as, for example, a display device, a speaker, a printing device, and/or the like, and/or an input component such as, for example, a microphone, a joystick, a satellite dish, a scanner, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a mobile computing device including a processor, memory, and a user interface, wherein the mobile computing device is programmed to:
compare a first set of pH levels measured using a chemical sensor during a first exercise regimen with a second set of pH levels measured using the chemical sensor during a second exercise regimen,
determine that the second exercise regimen results in a lower pH deviation than the first exercise regimen, and
recommend, via the user interface, the second exercise regimen.

2. The system of claim 1, further comprising: the chemical sensor, wherein the chemical sensor is a wearable device with needles sized for access to interstitial fluid and a chemical indicator positioned within the needles, wherein the chemical indicator changes optical properties in response to different pH levels.

3. The system of claim 2, wherein the mobile computing device includes an image sensor.

4. The system of claim 3, wherein the mobile computing device is programmed to: determine the first pH levels and the second pH levels based, at least in part, on an optical property of the chemical indicator.

5. The system of claim 1, wherein the mobile computing device includes an image sensor and is programmed to: determine the first pH levels and the second pH levels based, at least in part, on an optical property of a chemical indicator in a digital image.

6. The system of claim 1, further comprising: the chemical sensor, wherein the chemical sensor is part of an implantable medical device and comprises a chemical indicator, wherein the chemical indicator changes optical properties in response to different pH levels.

7. The system of claim 1, wherein the mobile computing device is programmed to: receive activity data measured using an acceleration sensor.

8. The system of claim 7, further comprising: the acceleration sensor coupled to a patient.

9. The system of claim 7, wherein the mobile computing device is programmed to: align in time pH data measured by the chemical sensor with the activity data.

10. The system of claim 7, wherein the mobile computing device is programmed to:
determine a pH-to-activity slope.

11. The system of claim 7, wherein the mobile computing device is programmed to: recommend a time period for starting the second exercise regimen based, at least in part, on the pH data and the activity data.

12. The system of claim 1, wherein the mobile computing device is programmed to:
align in time pH data measured using the chemical sensor with heart rate data;
determine that the heart rate data and the pH data indicate that a lactate threshold is reached due to musculoskeletal composition; and
recommend, via the user interface, another exercise regimen that increases muscle mass.

13. The system of claim 12, further comprising: a heart rate sensor coupled to a patient and configured to generate the heart rate data.

14. The system of claim 1, wherein the mobile computing device is programmed to:
align in time pH data measured using the chemical sensor with heart rate data;
determine that the heart rate data and the pH data indicate that exercise intolerance is due to a cardiovascular issue; and
recommend, via the user interface, another exercise regimen that improves cardiovascular response to exercise.

15. The system of claim 1, wherein the mobile computing device is programmed to:
compare a third set of pH levels measured using the chemical sensor during a third exercise regimen with the second set of pH levels,
determine that the second exercise regimen results in a lower pH deviation than the third regimen, and
recommend, via the user interface, that the third exercise regimen not replace the second exercise regimen.

16. A method comprising:
comparing a first set of pH levels measured using a chemical sensor during a first exercise regimen with a second set of pH levels measured using the chemical sensor during a second exercise regimen;
determining that the second exercise regimen results in a lower pH deviation than the first exercise regimen; and
recommending that the second exercise regimen replace the first exercise regimen.

17. The method of claim 16, further comprising:
comparing a third set of pH levels measured using the chemical sensor during a third exercise regimen with the second set of pH levels;
determining that the second exercise regimen results in a lower pH deviation than the third regimen; and
recommending that the third exercise regimen not replace the second exercise regimen.

18. The method of claim 16, further comprising:
aligning in time pH data measured using the chemical sensor with heart rate data;
determining that the heart rate data and the pH data indicate that a lactate threshold is reached due to musculoskeletal composition; and
recommending another exercise regimen that increases muscle mass.

19. The method of claim 16, further comprising:
aligning in time pH data measured using the chemical sensor with heart rate data;
determining that the heart rate data and the pH data indicate that exercise intolerance is due to a cardiovascular issue; and
recommending another exercise regimen that improves cardiovascular response to exercise.

20. The method of claim 16, further comprising:
receiving or observing activity data measured using an acceleration sensor; and
recommending a time period for starting the second exercise regimen based, at least in part, on the pH data and the activity data.
